(19) United States
(12) Patent Application Publication
(10) Pub. No.: US 2002/0026163 A1
(43) Pub. Date: Feb. 28, 2002

(54) Male incontinence device, application tool, and methods of assembly

(76) Inventor: Reinhold Grundke, Burghausen (DE)

Correspondence Address: BAKER BOTTS LLP, C/O INTELLECTUAL PROPERTY DEPARTMENT, THE WARNER, SUITE 1300, 1299 PENNSYLVANIA AVE, NW, WASHINGTON, DC, 20004-2400, US

(21) Appl. No.: 09/803,652

(22) Filed: Mar. 12, 2001

Related U.S. Application Data
(60) Provisional application No. 60/189,049, filed on Mar. 14, 2000.

(51) Int. Cl. A61F 5/44

(52) U.S. Cl. 604/347; 604/349; 604/544

(57) ABSTRACT

The present invention relates to a device and method for managing uncontrolled urine release in males. The device is flexible and is worn externally on the penis. This external device may further be attached to a urine collector, such as a bladder bag, and may be worn by a male person while standing, walking, sitting, or laying down. This urine drainage device comprises a body member and a sleeve member. The present invention further relates to an application aid for assembling the urine drainage device and for applying the assembled urine drainage device to a patient. The present invention further relates to an undergarment which may be worn with a urine drainage device.

### FIELD OF THE INVENTION

The invention concerns a device for the drainage of uncontrolled urine release in males. The invention further concerns devices which assist in the assembly and application of the male incontinence device and methods of such assembly and application.

### BACKGROUND OF THE INVENTION

Lack of urinary control (incontinence) is an ailment which affects approximately two million males in the United States alone. Solutions to managing uncontrolled urination, however, are limited. For some people, certain prescription medications assist in bodily control of urine release. In cases where medications do not work, are impracticable, or are only partially successful at controlling urine release, either a catheter is inserted into the bladder to drain urine into a collection receptacle, or the incontinent person is diapered.

Both catheter use and diapering have disadvantages. The insertion and removal of a catheter requires skill and is most commonly performed by a medical practitioner, requiring a person to remain in a hospital, nursing home, or other managed care setting. Catheters have to be removed and replaced approximately every five days resulting in high costs for people who rely on permanent assistance with urine drainage. Catheters are uncomfortable and the continuous insertion and removal of catheters irritates the patient's skin and may cause strictures and abscesses to form. Furthermore, catheters run a risk of causing urinary tract and bladder infections. Such infections are dangerous and can even become life threatening in an aging or feeble person.

Diapers, on the other hand, require changing approximately seven times per day also resulting in high costs. Many diapers can be seen through clothing, are bulky and uncomfortable for the wearer, and have the potential for leakage thereby soiling the wearer's clothing. Furthermore, because urine is alkaline, continuous contact between the delicate skin of the genital area and a urine soaked diaper leads to various rashes, skin maceration, and in extreme cases, formation of ulcers.

One alternative to internal catheters and diapering that has been used to drain urine release in males is an external catheter. Generally, an external catheter may be in the form of a sheath that is worn over a penis, the distal end of the sheath being connected to a tube that leads to a urine collection receptacle. The sheath is usually made of an elastic material, such as latex, and has an adhesive on the inside layer to adhere it to the penis during wear. Such external catheters are known as condom catheters due to their resemblance to condoms.

External male catheters are prone to several problems including leakage, becoming dislodged or pulled loose, and irritation or ulceration of the penile skin. For example, the presence of adhesive on the inside of the sheath can make removal of the catheter painful and can irritate to the delicate skin of the wearer. Furthermore, many of the current condom catheters provide inadequate drainage, or cannot be thoroughly cleaned. Prolonged exposure of delicate skin to urine may cause severe irritation. Also, the adhesive on the inside of a typical condom catheter may cause great pain and injury to the skin if the penis swells or becomes enlarged. This creates discomfort to the wearer and may constrict and injure the penile tissue which is attached to the adhesive.

Furthermore, most condom catheters are rolled, much like a condom. Removal of the catheter is accomplished by grasping the proximal end and pulling away from the body to invert the catheter. During removal, especially during removal by someone other than the wearer such as medical personnel or caretakers, the skin and surrounding body hair may be inadvertently pinched while trying to grasp the proximal end of the catheter.

A need, therefore, exists for an improved device for managing uncontrolled urine release which is inexpensive, runs a low risk of causing infections or skin irritations, and is comfortable for a male person to use.

### SUMMARY OF THE INVENTION

A device for the drainage of urine release in incontinent males is disclosed. In one embodiment, the device comprises a body member and a sleeve member. The body member may have a cylindrical section with a distal end and a proximal end (which goes next to the wearer's body). The distal end of the cylindrical section may have a narrowing section which narrows toward a spout member. The spout member may further be attached to a hose, tube, or other connection that connects the device to a bladder bag or other receptacle suitable for collecting urine. The proximal end of the cylindrical section may have at least one sealing ring. The proximal end of the cylindrical section optionally may have a plurality of flaps protruding outwardly from the proximal end.

The sleeve member may have a cylindrical portion with a distal end and a proximal end. The sleeve member comprises at least one inner sealing ring for engagement with the at least one sealing ring of the body member and a plurality of handles which aid in installing the sleeve member over the body member. The urine drainage device of the present invention is preferably made of a soft and pliable material such as latex, polyurethane, or silicon. The urine drainage device of the present invention may be assembled and applied using an application aid as described herein.

In one embodiment of the present invention, an application aid may comprise a frustoconical member having a wide end and a narrow end. The frusto-conical member may be smooth and may be made of any solid material such as a non-reactive metal, plastic, or wood. The wide end of the application aid may have a slot for engaging a urine drainage device of the present invention during application.

In one embodiment, a method of assembling a urine drainage device of the present invention may comprise placing a body member over a narrow end of a frusto-conical member of an application aid, so that the plurality of flaps of the body member face toward the wide end of the frusto-conical member. A sleeve member may be connected to the body member by pulling the plurality of handles of the sleeve member to stretch the sleeve member at least enough to slide it over the body member.

In one embodiment, a method of applying a urine drainage device of the present invention may comprise the steps of holding an assembled urine drainage device so that the proximal end of body member is facing a penis, grasping the plurality of flaps of the body member to widen the body member and sleeve member, inserting a penis into the body member, grasping the handles of the sleeve member to widen it and pull it over to cover the flaps of the body member.

An advantage of the present invention is that it provides an external urine drainage device for males which is comfortable and easy to use, avoids soiling of laundry when applied correctly, runs a low risk of causing urinary infections or skin irritation, provides the wearer with a large range of mobility, and is re-usable.

The tongue and sleeve member system of the urine drainage device of the present invention provide leakage protection while reducing the risk of skin irritation or penile tissue damage. In an embodiment where the body member contains flaps, the sleeve member covers the flaps and lays them against the penis in order to hold the device and prevent slipping and leakage. The flaps allow the device to bend if the penis swells, thus preventing pulling and irritation and constriction of blood flow.

The urine drainage device of the present invention is both environmentally friendly and reusable. It can be washed on a daily basis in warm water either by hand or in a washing machine. In one embodiment, the body member of the device comprises a rinsing aperture that allows the interior of the device to be cleaned without necessitating removal of the device from the wearer. Simple washing with or without a mild soap or detergent prevents the formation of biological or crystalline deposits on the device and helps reduce the risk of infection and/or skin irritation. The ability to wash the device without removing it from the wearer reduces potential skin irritation from the repeated removal and application of the device.

The present invention is further directed to an undergarment such as a pair of briefs, which may be worn with a urine drainage device of the present invention, or any other appropriate urine drainage device. The undergarment may comprise a front panel and a back panel. The front panel may have a hole and a fastener for inserting and securing a urine drainage device, such as described above. An undergarment of the present invention allows a urine drainage device to be worn comfortably while providing the wearer with additional secured attachment of the urine drainage device.

Other advantages and embodiments of the invention are set forth in part in the description which follows, and in part, will be obvious from this description, or may be learned from the practice of the invention.

### DESCRIPTION OF THE DRAWINGS

The design of the urine drainage device is explained in detail by drawings, showing:

FIG. 1 A side view of a body member of a urine drainage device of the present invention.

FIG. 2 A side view of a sleeve member of a urine drainage device of the present invention.

FIG. 3 A top view of a sleeve member of a urine drainage device of the present invention.

FIG. 4 A side view of an application aid of the present invention.

FIG. 5 A diagram depicting a method for assembling a urine drainage device of the present invention using an application aid of the present invention.

FIG. 6 An assembled urine drainage device of the present invention depicted on an application aid of the present invention.

FIG. 7 A bottom view of an assembled urine drainage device of the present invention prior to the device being applied to a user.

FIG. 8 A pair of briefs which may be used with a urine drainage device of the present invention.

FIG. 9 A side view of a body member of a urine drainage device of the present invention, similar to the body member of FIG. 1, but without flaps.

FIG. 10 A side view of a sleeve member of a urine drainage device of the present invention, which member contains four handles.

FIG. 11 A top view of the sleeve member of FIG. 10.

FIG. 12 A side view of a sleeve member of a urine drainage device of the present invention.

### DETAILED DESCRIPTION OF THE INVENTION

As embodied and broadly described herein, the present invention is directed to a novel system for managing uncontrolled urination (incontinence) in males. The present invention is particularly directed to washable and re-usable external devices which can be comfortably worn by male subjects and which avoid soiling of laundry, run a low risk of causing urinary infections or skin irritation and allow a male subject to remain mobile while wearing the device.

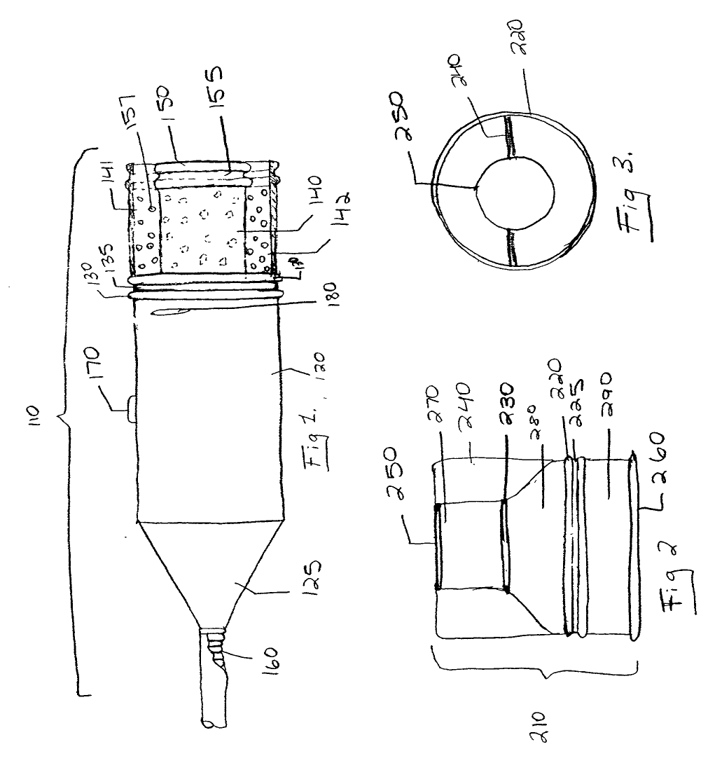

As depicted, in whole or in part, in FIGS. 1, 2, 3 and 7, one embodiment of a urine drainage device of the present invention comprises a body member 110 and a sleeve member 210. The body member has a cylindrical section 120 with a distal end and a proximal end. The distal end of the cylindrical section 120 has a narrowing section 125 which narrows toward a spout member 160. In a preferred embodiment, the narrowing section 125 is funnel shaped to prevent back flow of urine during urination, helping to minimize wetness and thereby to help prevent irritation and infection which may be caused by prolonged exposure to urine.

In one embodiment, cylindrical section 120 may further comprise a rinsing aperture 170. Rinsing aperture 170 allows the interior of cylindrical section 120 to be rinsed and cleaned without removing the urine drainage device from the wearer. In a preferred embodiment, a rinsing aperture is formed to permit the rinsing aperture to be coupled to a rinsing device, e.g., a syringe, which is capable of supplying a cleaning solution, such as warm water mixed with a mild detergent. When not in use, the rinsing aperture 170 may be filled with a removable stopper (not shown).

In another embodiment, cylindrical section 120 may further optionally comprise at least one ventilation opening 180, and preferably near the proximal end. Ventilation opening 180 helps damp air to escape thereby reducing the risk of skin rashes, and improves the draining of urine into spout member 160 by relieving any vacuum formation within body member 110.

In another embodiment, cylindrical section 120 may optionally comprises a one-way valve to assist in ventilation and to prevent any vacuums from forming within body member 110. The one-way valve could be installed in either ventilation opening 180 or in rinsing aperture 170, or it could be installed in its own opening.

The proximal end of cylindrical section 120 has at least one sealing ring 130. In a preferred embodiment, cylindrical section 120 comprises two outer sealing rings 130 separated from one another by a furrow 135. The two outer sealing rings 130 and furrow 135 are used to secure sleeve member 210 about body member 110. The proximal end of cylindrical section 120 may optionally further comprises a plurality of a (see, e.g., flaps 140, 141, and 142) protruding from said proximal end. In one embodiment, the flaps have at least one outer sealing ring 150 for further engagement with sleeve member 210. In a preferred embodiment, the urine drainage device of the present invention comprises three flaps, 140, 141, and 142, and the interiors of the flaps have a plurality of protrusions 157 to improve adhesion of the flaps to the penis. In another preferred embodiment, the plurality of flaps, 140, 141, and 142, each have at least one finger slot 156 as depicted in FIG. 7. Finger slots 156 aid the patient, or person assisting the patient, in applying the urine drainage devise to the wearer by providing a place wherein a finger may be placed in order to help widen the proximal end of the device for placement on a patient.

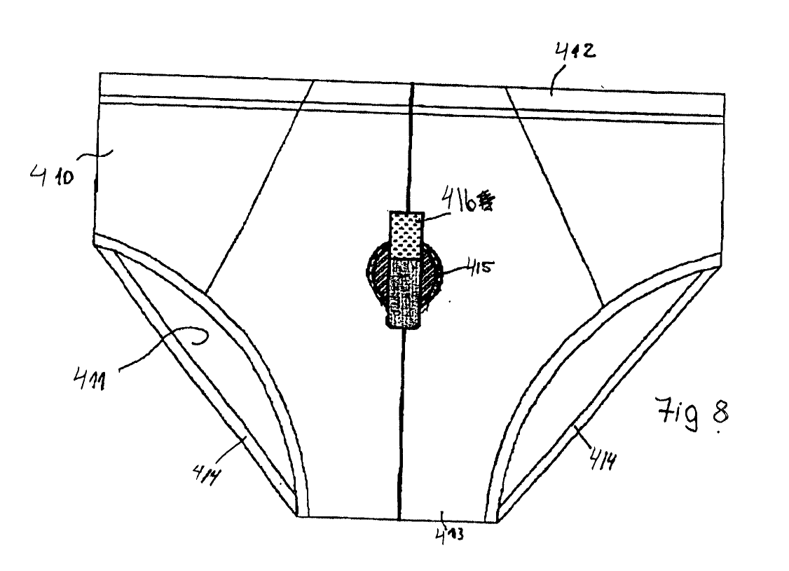

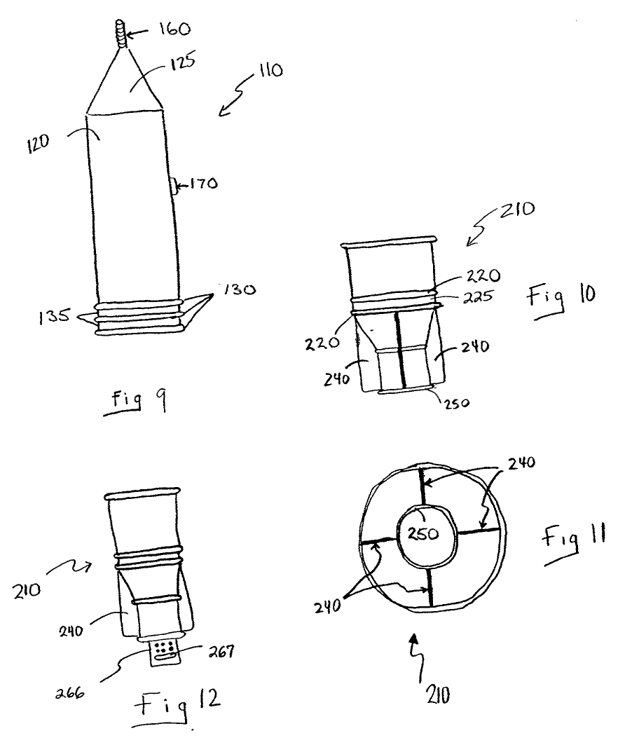

An embodiment of the body member 110 without flaps is shown in FIG. 8. As shown in FIG. 8, the body member 110 comprises three sealing rings 130 and two furrows 135, which could be aligned with the sealing rings located on sleeve member 210 illustrated in FIGS. 10 and 11. The embodiment of the sleeve member 21 0 shown in FIGS. 9 and 10 comprises two sealing rings 220, a furrow 225, and four handles 240

A “sealing ring” is, generally, an annular ridge which helps to reduce the likelihood of urine leakage from the device of the present invention. The annular ridge need not be continuous (see, e g, the sealing ring of the flaps disclosed below), though it certainly may be a continuous ridge (see, e.g., sealing ridge 260 disclosed below). Further, a sealing ring may function in conjunction with other sealing rings (see, e.g., sealing rings 130 and 220), or may function in isolation (see, e.g., sealing ridge 260 disclosed below). The terms “outer” and “inner” may be used in conjunction with “sealing rings” but are intended merely to suggest that a particular sealing ring is intended to function in conjunction with another sealing ring on an opposite, engaging member, and to this extent, the terms are not to be construed as limited a sealing ring formed on the inside or outside of a particular member.

Sleeve member 210 of the urine drainage device of the present invention may have a cylindrical portion with a proximal end (which goes next to the wearer) and a distal end. In a preferred embodiment, the cylindrical portion of sleeve member 210 comprises three segments; a first segment 270, a second segment 280, and a third segment 290. First segment 270 is preferably narrower in diameter than the third segments. Generally, the proximal end corresponds to first segment 270, and the distal end corresponds to third segment 290.

The distal end of the sleeve member 210 may comprise at least one sealing ring 260 to help reduce the likelihood of leakage. In FIG. 6, sealing ring 260 is operates in isolation, though it is contemplated that at least one sealing ring 260 could function in conjunction with matching sealing rings on body member 110.

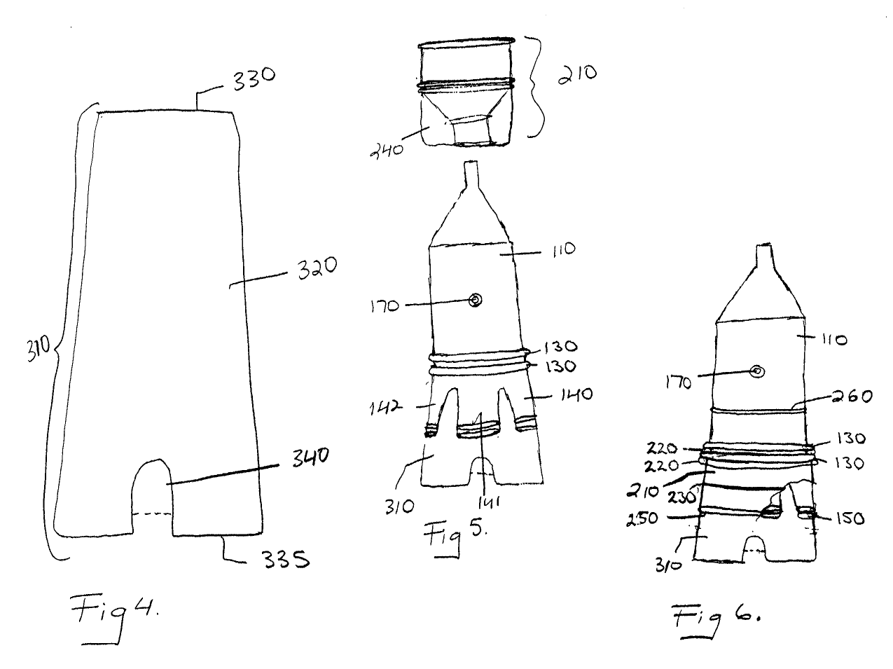

The proximal end of sleeve member 210 may comprise a plurality of handles 240 which aid in inserting sleeve member 210 over body member 110. In FIGS. 2 and 3, the plurality of handles 240 comprise two side tabs which can be griped by one's fingers and pulled apart to provide easier access to the inner diameter of the proximal end of sleeve member 210.

In one embodiment, first segment 270 may comprises at least one, and preferably, at least two, inner sealing rings 250 for engagement with the at least one sealing ring 150 and furrow 155 of flaps 140, 141, and 142 of body member 110. Second segment 280 may optionally comprise at least one sealing ring (not shown) to be mated with corresponding sealing rings (not shown) of body member 110. Third segment 290 preferably comprises a sealing ring 260 to provide additional resistance to leakage.

In one preferred embodiment of the present invention, sleeve member 210 comprises one sealing ring 220 and cylindrical section 120 comprises two sealing rings 130 separated from one another by a furrow 135, such that the sealing ring 220 of sleeve member 210 can rest within furrow 135 of cylindrical section 120, and such that the sealing ring 220 and the sealing rings 130 form a leak-resistant seal.

In another preferred embodiment of the present invention, sleeve member 210 comprises two sealing rings 220 (only one of which is shown), separated by a furrow (not shown) and cylindrical section 120 comprises one sealing ring 130, such that the sealing ring 130 of cylindrical section 120 can rest within the furrow (not shown) between the two sealing rings 220 of sleeve member 210, and such that the sealing rings 220 and the sealing ring 130 form a leak-resistant seal.

In another preferred embodiment of the present invention, sleeve member 210 comprises two sealing rings 220 (only one of which is shown), separated by a furrow (not shown) and cylindrical section 120 comprises two sealing rings 130 separated by a furrow 135, such that one of the sealing rings 130 can rest within the furrow (not shown) between the two sealing rings 220 and such that one of the sealing rings 220 can rest within furrow 135, thereby collectively forming a leak-resistant seal.

The number of sealing rings 220 and the number of sealing rings 130 may vary, but collectively, at least one furrow should form, on either of the body member or the sleeve member, and the at least one furrow should be filled by a sealing ring of the opposite member to form a leak-resistant seal. It is specifically contemplated, for example, that five furrows could be formed by six rings, and then be each furrow could be filled by a sealing ring formed on the opposite, engaging member. Further, the word “seal” should not be interpreted to require an air-tight seal or a water-proof seal, but merely a boundary which resists leaks.

In one preferred embodiment, the thickness of the second segment 280 is preferably thinner and more flexible than the first and third segments. For example, in one embodiment, the thickness of the second segment 280 is preferably one-fifth to one-half the thickness of the third segment, so that the second segment 280 has greater flexibility.

The urine drainage device of the present invention is preferably made of a soft and pliable material such as latex, polyurethane, silicon, or any other appropriate material common to those of skill in the art of external urine drainage devices. The body member 110 and sleeve member 210 of a urine drainage device may each be made of a different soft and pliable material. In a preferred embodiment, both body member 110 and sleeve member 210 are made of silicon. Body member 110, and sleeve member 210, may be made to any appropriate width and length to accommodate any size and shape of a wearer. In one embodiment, body member 110 and sleeve member 210 are formed as one piece.

A urine drainage device, such as that of the present invention, may be assembled and applied using an application aid 310 as depicted in FIG. 4, 5, and 6. In one embodiment of the present invention, an application aid may comprise a frusto-conical member 320 having a wide end 335 and a narrow end 330. In a preferred embodiment, the narrow end 330 is closed. In another embodiment, wide end 335 of frusto-conical member 320 has a slot 340 which may be used to aid a wearer, or a person assisting the wearer, in applying a urine drainage device to the wearer. In a preferred embodiment, frusto-conical member 320 is smooth, hollow, and is made of any sturdy material such as a non-reactive metal, plastic, wood, or any other appropriate material known to those of skill in the art. Frusto-conical member 320 may be a one piece member or may comprise several pieces. Frusto-conical member 320 may further be solid or may be made of a sturdy mesh, or any other appropriate construction. The outside surface of said frusto-conical member 320 should offer sufficient friction to prevent the body member 110 from slipping when the user is trying to place the sleeve member 210 over said body member 110.

Frusto-conical member 320 and slot 340 are shaped in such a fashion to accommodate the shapes of body member 110 and the rinsing aperture 170, respectively. When body member 110 is inserted into frusto-conical member 320, the rinsing aperture 170 can be easily aligned with slot 340 such that the application aid 310 can be used in applying the urine drainage device to a wearer.

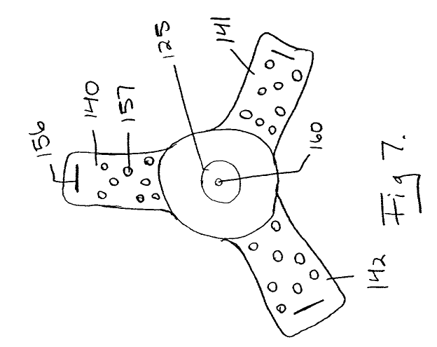

In one embodiment, a method of assembling a urine drainage device of the present invention may comprise placing body member 110 over narrow end 330 of frusto-conical member 320 of an application aid, so that the proximal end (that which contains sealing rings 130) of body member 110 face toward wide end 335 of frusto-conical member 320 as depicted in FIG. 5. Sleeve member 210 may be connected to body member 110 by pulling the plurality of handles 240 of sleeve member 210 to stretch sleeve member 210 at least enough to slide it over body member 110. In a preferred embodiment, at least one inner sealing ring 220 and furrow 225 of sleeve member 210 is then engaged with at least one sealing ring 130 and furrow 135 of body member 110 to connect sleeve member 210 to body member 110. A urine drainage device of the present invention may also be assembled using any other appropriate means including, but not limited to, rolling sleeve member 210 onto cylindrical section 120, placing body member 110 onto the penis of a wearer, then unrolling sleeve member 210 onto the proximal end of body member 110 to seal the urine drainage device and bold it in place.

In another embodiment, a method of applying a urine drainage device of the present invention using application tool 310 may comprise the following steps: assembling a urine drainage device using one of the methods previously described; inserting the narrowing section 125 of body member 110 of the assembled urine drainage device into the frusto-conical member 320 of application tool 310 through the wide end 335, such that the proximal end of the ruing drainage device is located at the wide end 335 of the application tool 310, and in the case of a urine drainage device which contains a rinsing aperture 170, such that the rinsing aperture 170 is aligned with slot 340 of the application tool; positioning the application tool towards a patient such that the proximal end of body member 110 is facing the patient's penis; widening the proximal end of the assembled urine drainage device; and placing the urine drainage device over the patient's penis. If the body member 110 contains a plurality of flaps, e.g., flaps 140, 141, and 142, then the step of widening may be accomplished by grasping the flaps and opening the proximal end of the urine drainage device. If the body member 110 contains finger slots, e.g., finger slots 156, the step of widening may comprise inserting fingers into the finger slots and pulling on the finger slots to widen the proximal end of the assembled urine drainage device.

In a preferred embodiment, at least one inner sealing ring 230 of sleeve member 210 is engaged with at least one sealing ring 150 and furrow 155 of a plurality of flaps 140, 141, and 142 of body member 10 to further connect sleeve member 210 with body member 110 and to cover the plurality of flaps and adhere them to the patient. Fitting the sealing rings within the furrow may be accomplished after the urine drainage device has been secured to the patient. This flap and sleeve member system of the urine drainage device of the present invention provides leakage protection while reducing the risk of skin irritation or penile tissue damage. Sleeve member 210 covers the plurality of flaps (i.e. 140, 141, and 142) and secures them against the penis in order to help hold the device and prevent slipping and leakage. The flaps allow the device to bend to help prevent irritation and constriction of blood flow.

A urine drainage device of the present application may further be attached, via spout member 160, to a urine collector, such as a bladder bag, which may be worn on the leg. A urine drainage device of the present invention may thus be worn by a male person while standing, walking, sitting, or laying down. Spout member 160 may be attached directly to a urine collector or it may be attached through a flexible tube. A urine collector is preferably attached to a urine drainage device of the present invention after the urine drainage device is applied to the patient.

The present invention is further directed to an undergarment, such as a pair of sport briefs as depicted in FIG. 8, which may be worn with a urine drainage device of the present invention, or any other appropriate urine drainage device. An undergarment may comprise a front panel 410 and a back panel 411. Front panel 410 may have a hole 415 for insertion of a penis and at least one fastener 416 for attachment of a urine drainage device.

In a preferred embodiment, a pair of sport briefs of the present invention comprise a front panel 410 (which also contains a middle panel 413), a back panel 411, an elastic waistline 412, and hemmed elastic leg holes 414. Middle panel 413 has a hole 415 and at least one fastener 416. At least one fastener 416 may be any system of snaps, buttons, hook and loop fastening material (such as VELCRO), or any other appropriate system or combination thereof. In a preferred embodiment, at least one fastener 416 is a hook and loop fastening material which attaches to the urine drainage device to give the wearer greater confidence that the urine drainage device will not come loose while wearing the device. To be worn with the briefs, a urine drainage device must be modified to accommodate the fastener on the briefs. For example, a urine drainage device could be modified to contain an opening through which the fastener 416 could pass to secure the device to the briefs. The slot (not shown) could be formed an extension of one or more of the flaps (140, 141, and 142, disclosed herein) or could be formed on the sleeve member by attaching an extension to sealing ring 250, or both. FIG. 12 depicts an embodiment of the sleeve member 210 which comprises extension tab 266, which extension tab includes an opening 267 which could accommodate the fastener 416. Extension tab 266 could also act as a handle in a similar fashion to handle 240. In much the way that sealing ring 250 can have an extension, one of the flaps (140, 141 or 142) could be formed with an extension to contain, for example, a finger slot 156, which could serve the dual purpose of a finger slot and a handle. One such embodiment is shown in FIG. 7.

In a further preferred embodiment, a sport brief of the present invention is made of cotton, a cotton/polyester blend, or any other comfortable material commonly used for making undergarments. In another embodiment, an undergarment of the present invention is made of paper or some other type of material suitable for making the undergarment economically disposable. An undergarment of the present invention allows the urine drainage device to be worn comfortably while providing the wearer with additional secured attachment of a urine drainage device.

Other embodiments and uses of the invention will be apparent to those skilled in the art from consideration of the specification and practice of the invention disclosed herein. The specification and examples should be considered exemplary only with the true scope and spirit of the invention indicated by the following claims. As will be easily understood by those of ordinary skill in the art, variations and modifications of each of the disclosed embodiments can be easily made within the scope of this invention as defined by the following claims.

What is claimed is:

1. A system for controlling urine release in incontinent males, the system comprising:
a body member comprising a cylindrical section with a distal end and a proximal end, said distal end having a narrowing section which narrows toward a spout member, and said body member having a first number of sealing rings near the proximal end of the cylindrical section, said first number of sealing rings being at least one; and
a sleeve member comprising a tubular portion with a distal end and a proximal end, wherein the sleeve member comprises second number of sealing rings near the distal end of the sleeve member, said sleeve member having a plurality of handles, and said second number of sealing rings being at least one;
wherein said proximal end of the body member is formed to align with the distal end of the sleeve member, and wherein at least one of the first number and the second number is greater than two, such that at least two sealing rings are formed adjacent to each other on one member to form a furrow therebetween, which furrow can accommodate a sealing ring of the other member to form a leak resistant seal.

2. The system of claim 1 wherein the body member comprises at least two sealing rings separated by a furrow and the sleeve member comprises at least one sealing ring, such that the sealing ring of the sleeve member can be positioned within the furrow of the body member to form a leak resistant seal.

3. The system of claim 1 wherein the body member comprises at least one sealing ring and the sleeve member comprises at least two sealing rings separated by a furrow, such that the sealing ring of the body member can be positioned within the furrow of the sleeve member to form a leak resistant seal.

4. The system of claim 1 wherein the body member further comprises at least one flap

5. The system of claim 4 wherein the sleeve member further comprises at least one sealing ring on the proximal end of the sleeve member, and wherein each of said at least one flaps has at least two sealing rings separated by a furrow, such that the sealing ring of the proximal end of the sleeve member can be placed within the furrow on each of said at least one flaps.

6. The system of claim 1 wherein the handles are formed along a side of the proximal end of the sleeve member.

7. The system of claim 1 wherein the sleeve member further comprises a tubular middle section between the distal and the proximal ends of the sleeve member, said tubular middle section having a thickness which is substantially less than the thickness of said distal end and the proximal ends of the sleeve member, such that said middle section exhibits greater flexibility and softness than said distal and proximal ends.

8. The system of claim 1 wherein the plurality of flaps have a plurality of protrusions on their interior surface and at least one of said flaps has at least one opening therein.

9. The system of claim 1 wherein the body member further comprises a rinsing aperture, and wherein the system further comprises a removable stopper which can be placed within the rinsing aperture.

10. The system of claim 7 wherein the removable stopper contains a one-way valve.

11. The system of claim 1 wherein the body member further comprises at least one ventilation opening.

12. The system of claim 1 wherein said sleeve member further comprising a tab connected to proximal end of said sleeve member, said tab having an opening.

13. The system of claim 1 further comprising an undergarment having a fastener for securing either the body member or the sleeve member to the undergarment.

14. The system of claim 1 further comprising an application aid which can be used to assemble a urine drainage device comprising the body member and the sleeve member, said application aid comprising a frusto-conical member having a narrow end and a wide end.

15. A urine drainage device comprising:
a body member comprising a cylindrical section with a distal end and a proximal end, said distal end having a narrowing section which narrows toward a spout member, and said body member having a first number of sealing rings near the proximal end of the cylindrical section, said first number of sealing rings being at least one; and
a sleeve member comprising a tubular portion with a distal end and a proximal end, wherein the sleeve member comprises second number of sealing rings near the distal end of the sleeve member, said sleeve member having a plurality of handles, and said second number of sealing rings being at least one;
wherein said proximal end of the body member is formed to align with the distal end of the sleeve member, and wherein at least one of the first number and the second number is greater than two, such that at least two sealing rings are formed adjacent to each other on one member to form a furrow therebetween, which furrow can accommodate a sealing ring of the other member to form a leak resistant seal.

16. The device of claim 15 wherein the body member comprises at least two sealing rings separated by a furrow and the sleeve member comprises at least one sealing ring, such that the sealing ring of the sleeve member can be positioned within the furrow of the body member to form a leak resistant seal.

17. The device of claim 15 wherein the body member comprises at least one sealing ring and the sleeve member comprises at least two sealing rings separated by a furrow, such that the sealing ring of the body member can be positioned within the furrow of the sleeve member to form a leak resistant seal.

18. The device of claim 15 wherein the body member further comprises at least one flap.

19. The device of claim 18 wherein the sleeve member further comprises at least one sealing ring on the proximal end of the sleeve member, and wherein each of said at least one flaps has at least two sealing rings separated by a furrow, such that the sealing ring of the proximal end of the sleeve member can be placed within the furrow on each of said at least one flaps.

20. The device of claim 15 wherein the handles are formed along a side of the proximal end of the sleeve member.

21. An application aid for assembling and applying a urine drainage device, the application aid having:
a frusto-conical member comprising a narrow end and a wide end;
and a slot formed in the wide end of said frusto-conical member;
said frusto-conical member being shaped to accommodate a body member of a urine drainage device and said frusto-conical member having an outer surface being shaped to offers sufficient friction to prevent said body member from slipping when installed over said frusto-conical member; and
said frusto-conical member and said slot being shaped to accommodate and receive a urine drainage device when inserted in said application aid.

23. The application aid of claim 21 wherein the frusto-conical member is hollow and is made of a material selected from the group consisting of wood, plastic, non-reactive metal, or a combination thereof.

24. An undergarment for use with a urine drainage device, the undergarment comprising:
a front panel;
a rear panel connected to said front panel so as to form a waist line and two leg openings;
said front panel comprising
an opening wherein a penis could pass through to be inserted into the urine drainage device; and
at least one fastener to secure said urine drainage device to said undergarment.

25. The undergarment of claim 24 wherein the at least one fastener is selected from the group consisting of snaps, buttons, hook and loop fastening material and a combination thereof.

26. A method of using an application tool with a urine drainage device, said application tool having a frusto-conical member with a narrow end and a wide end, said method comprising:
placing a body member of the urine drainage device over the narrow end of the frusto-conical member, so that a proximal end of the body member faces toward the wide end;
stretching a proximal end of a sleeve member so as to form an opening at least as wide as the narrow end of the frusto-conical member;
placing said sleeve member over the body member, all over the frusto-conical member, so that a distal end of the sleeve member covers the proximal end of said body member;
removing said body member and said sleeve member by slipping said frusto-conical member out from under the overlapping body and sleeve members and such that an assembled urine drainage device is formed.

27. The method of claim 26 further comprising:
holding said application tool such that the assembled urine drainage device may be inserted within said application tool;
placing said assembled urine drainage device within said application tool such that the proximal end of the sleeve member is closest to the wide end of the application tool;
orienting the wide end the application tool towards a penis;
and stretching the proximal end of the sleeve member so as permit the assembled urine drainage device to be placed over the penis.

28. The method of claim 26 wherein the stretching step is accomplished by pulling a plurality of handles formed on the sleeve member at least enough to slide it over the narrow end of the application tool and the body member resting on said application tool.

29. The method of claim 26 wherein the step of placing the sleeve member over the body member is accomplished by placing said sleeve member over the body member, all over the frusto-conical member, so that a distal end of the sleeve member covers the proximal end of said body member and so that a sealing ring of said sleeve member fits within a furrow of said body member so as to form a leak resistant seal.

30. The method of claim 27 wherein the step of placing the assembled urine drainage device within said application tool is accomplished by aligning a rinsing aperture of the assembled device with a slot on the wide end of the frusto-conical member and then inserting the assembled device such that the proximal end of the sleeve member is closest to the wide end of the application tool.
